(19) United States
(12) Patent Application Publication
(10) Pub. No.: US 2007/0276242 A1
(43) Pub. Date: Nov. 29, 2007

(54) System And Method For Localized Measurement And Imaging Of Viscosity Of Tissues

(76) Inventor: Elisa E. Konofagou, New York, NY (US)

Correspondence Address: BAKER BOTTS L.L.P., 30 ROCKEFELLER PLAZA, 44TH FLOOR, NEW YORK, NY, 10112-4498, US

(21) Appl. No.: 11/697,579

(22) Filed: Apr. 6, 2007

Related U.S. Application Data
(63) Continuation of application No. PCT/US05/37670, filed on Oct. 17, 2005.
(60) Provisional application No. 60/619,136, filed on Oct. 15, 2004.
(60) Provisional application No. 60/619,636, filed on Oct. 18, 2004.
(60) Provisional application No. 60/717,864, filed on Sep. 16, 2005.

(51) Int. Cl. A61B 8/00

(52) U.S. Cl. 600/437

(57) ABSTRACT

A system and method for imaging the localized viscoelastic properties of tissue is disclosed. An oscillatory radiation force is applied to tissue in order to induce a localized oscillatory motion of the tissue. The phase and amplitude of the induced localized oscillatory motion of the tissue is also detected while the oscillatory radiation force is being applied. The viscous properties of the tissue are determined by a calculation of a phase shift between the applied oscillatory radiation force and the induced localized oscillatory motion of the tissue. The oscillatory force force inducing local oscillatory motion may be a single amplitude modulated ultrasound beam.

### CLAIM FOR PRIORITY TO RELATED APPLICATIONS

This application claims the benefit of U.S. Provisional Patent Application Ser. No. 60/619,136, filed on Oct. 15, 2004, entitled “System and Method for Localized Measurement and Imaging of Viscosity of Tissues,” U.S. Provisional Patent Application Ser. No. 60/619,636, filed on Oct. 18, 2004, entitled “System and Method of Localized Measurement and Imaging of Viscosity of Tissues,” U.S. Provisional Patent Application Ser. No. 60/717,864, filed on Sep. 16, 2005, entitled “Single-Element Focused Transducer and Method for Harmonic Imaging,” all of which are hereby incorporated by reference in their entirety herein.

### BACKGROUND

1. Field of the Invention

This invention relates to an imaging technique and system that uses an oscillatory radiation force to measure the characteristics of tissues of a patient, and more particularly to a technique and system for simultaneously measuring the viscosity of such tissues of a patient by comparing the amplitude and phase of the localized tissue displacement in response to the applied oscillatory radiation force.

2. Background of the Related Art

Medical practitioners have long used palpation as a diagnostic tool The health care provider touches and feels the patient's body part with his or her hands to examine the size, consistency, texture, location, and tenderness of the organ or body part to detect the presence of abnormalities which could indicate pathologies. This technique is typically quite effective because the mechanical properties of diseased tissue are typically different from those of normal tissue surrounding therm. For example, breast cancers have long been known to be harder than benign nodules at palpation. Palpation, however, is limited to the detection of tumors that are close to the skin surface. In addition, other properties have been associated with diseased tissue, such as water content, tissue density, and viscosity, which are not amenable to precise detection by palpation techniques alone.

Elasticity imaging techniques have been developed to detect the mechanical characteristics of tissues without the need for manual palpation. One method which induces vibration remotely to detect such tissue properties is ultrasound-stimulated acoustic emission imaging as described in Fatemi, M. and Greenleaf J F, “Ultrasound-Stimulated Vibro-Acoustic Spectrography,” Science 1998; 280(5360):82-85 (hereinafter “Fatemi and Greeleaf”), which is incorporated by reference in its entirety herein. This method uses ultrasound-induced radiation force to probe tissue properties. As an ultrasound beam propagates through tissue, part of its energy is absorbed and part of it scattered away. The momentum change of the beam results in a force that acts on the tissue. According to this technique, in which the ultrasound beams are operating at slightly different frequencies (f₁ and f₂, f₁≈f₂), the beams overlap at the focal region where the waves interfere and generate a wave that is amplitude-modulated by their difference frequency (f_{d}=f₂−f₁). An object at the overlapping zone experiences an average energy density <E> that fluctuates at the frequency of f_{d}. This varying force causes the tissue to move at frequency f_{d} and, thus, generates an acoustic source.

The magnitude of the acoustic wave emitted by the source depends on the radiation force and the mechanical frequency response of the tissue at the frequency of f_{d}. The stimulated acoustic signal propagates through the tissue and can be detected by an external hydrophone (see, e.g., Fatemi and Greenleaf, above, and Konofagou E E, Thierman J, Kajalainen T., Hynynen K., “The Temperature Dependence of Ultrasound-Stimulated Acoustic Emission,” Ultrasound Med Biol. 2002; 28(3): 331-338, both of which are which are incorporated by reference herein.) The resulting acoustic signal, however, is a combination of the mechanical and acoustical properties of the tissue, the resonance characteristics of the transducer housing and its surroundings, and its interaction at the hydrophone. Therefore, stiffness estimation using this method is extremely challenging.

To avoid the artifacts and drawbacks of the ultrasound stimulated acoustic emission application, an improvement referred to as Harmonic Motion Imaging (HMI) was proposed in Konofagou E. and Hynynen K., “Localized Harmonic Motion Imaging: Theory, Simulations, and Experiments,” Ultrasound Med Biol. 2003; 29(10): 1405-1413 (hereinafter “Konofagou and Hynynen”), which is incorporated by reference in its entirety herein. Konofagou and Hynynen proposed utilization of the radiation force of the overlapping ultrasound beams, but also to use a separate ultrasound beam to probe the induced tissue motion. The amplitude as well as the frequency content, of this motion, provides information about the mechanical properties of the tissue.

Most elasticity imaging techniques make the assumption that the tissues are purely elastic so that the measured mechanical response can be more directly associated to the elastic modulus of the tissue. The HMI technique, discussed in Konofagou and Hynynen above, applies an oscillatory radiation force in a small tissue region (on the order of a beamwidth) and images the resulting localized harmonic displacement to detect the elastic modulus of tissue structures. However, the assumption of purely elastic tissue properties does not always provide optimal results, as all tissues are actually viscoelastic (see, e.g., Fung Y. C., Biomechanics, Second Ed., Springer-Verlag, New York, 1993).

Accordingly, there is a need in the art for an elasticity imaging technique which is able to evaluate localized viscosity characteristics of tissues being studied.

### SUMMARY OF THE PRESENT INVENTION

It is an object of the current invention is to overcome the aforementioned limitations to provide a viscoelastic imaging technique.

It is another object of the current invention to provide the application of radiation force on the same tissue region and a simpler transducer design.

In accordance with an embodiment of the present invention, a method for imaging the localized viscoelastic properties of tissue is provided comprising receiving a first signal representative of an applied oscillatory radiation force having a phase and amplitude, receiving a second signal representative of an induced localized oscillatory motion of the tissue induced by the application of the oscillatory radiation force, the second signal having a phase and amplitude, and determining the viscous properties of the tissue by calculation of a phase shift between the applied oscillatory radiation force and the induced localized oscillatory motion of the tissue. In some embodiments, receiving the second signal representative of an induced localized oscillatory motion of the tissue includes determining the axial displacements of tissue from successive images of the tissue.

In certain embodiments, the method includes, prior to receiving the first signal, inducing localized oscillatory motion of tissue. The method may further include applying an oscillatory ultrasound radiation force. In some embodiments, the method may include applying two overlapping focused ultrasound beams. In certain embodiments, the method may include applying one focused ultrasound beam. The method may further include applying one amplitude modulated ultrasound beam.

A system for imaging the localized viscoelastic properties of tissue is provided comprising a processor and a memory operatively coupled to the processor, the memory storing program instructions for execution by the processor to receive a first signal representative of an applied oscillatory radiation force having a phase and amplitude, to receive a second signal representative of an induced localized oscillatory motion of the tissue induced by the application of the oscillatory radiation force, the second signal having a phase and amplitude, and to determine the viscous properties of the tissue by calculation of a phase shift between the applied oscillatory radiation force and the induced localized oscillatory motion of the tissue. In some embodiments, the processor is further adapted to determine axial displacements of tissue from successive images of the tissue.

The system may further include a first transducer for inducing localized oscillatory motion of tissue through the application of the oscillatory radiation force. The first transducer applies one amplitude modulated ultrasound beam. The system may further comprise a second transducer detecting a phase and amplitude of the induced localized oscillatory motion of the tissue simultaneous with the application of the oscillatory radiation force.

In accordance with the invention, the object of providing a system and method for measuring the stiffness and viscosity of tissues through the application of harmonic load applied to a small tissue region has been met. Further features of the invention, its nature and various advantages will be apparent from the accompanying drawings and the following detailed description of illustrative embodiments.

### BRIEF DESCRIPTION OF THE DRAWINGS

FIG. 1 is a block diagram of a model for characterizing viscoelastic properties, as is known in the art.

FIG. 2 is a plot illustrating phase shift as a function of frequency in accordance with the present invention.

FIG. 3 is a time vs. amplitude plot illustrating tissue displacement as a result of the application of a localized harmonic force, in accordance with the present invention.

FIG. 4 is a time vs. amplitude plot illustrating phase shift of tissue displacement, in accordance with the present invention.

FIGS. 5(a)-5(b) are representations of acoustic radiation force fields taken at periodic intervals produced by two overlapping ultrasound beams in accordance with the present invention.

FIG. 6 is a schematic representation of a system in accordance with an exemplary embodiment of the present invention.

FIGS. 7(a)-7(b) are representations of acoustic radiation force fields taken at periodic intervals produced by one ultrasound beam in accordance with the present invention.

FIG. 8(a) is a time plot representing a periodic force for local application to tissue in accordance with the present invention.

FIG. 8(b) is a time plot representing a lower frequency force for modulation of the force represented in FIG. 8(a) in accordance with the present invention.

FIG. 8(c) is a time plot representing the amplitude modulated signal output of the function generator in accordance with the present invention.

FIG. 8(d) is time plot representing the normalized acoustic intensity generated at the focus of the medium in accordance with the present invention.

FIG. 9 is a time plot representing the normalized input radiation force intensity, the localized displacement of the tissue, and the phase shift thereof in accordance with the present invention.

FIG. 10(a) is a plot representing tissuse displacement as a function of stiffness in accordance with the present invention.

FIG. 10(b) is a plot representing phase shift as a function of stiffness in accordance with the present invention.

FIG. 11 is a representation of a medium have an inclusion of different stiffness in accordance with the present invention.

FIG. 12(a) is 2D representation of displacement of the medium illustrated in FIG. 11 in accordance with the present invention.

FIG. 12(b) is 2D representation of phase shift of the medium illustrated in FIG. 11 in accordance with the present invention.

FIG. 13(a) is a plot representing displacement of the medium as a function of sonication time in accordance with thre present invention.

FIG. 13(b) is a plot representing phase shift as a function of sonication time in accordance with thre present invention.

Throughout the figures, the same reference numerals and characters, unless otherwise stated, are used to denote like features, elements, components or portions of the illustrated embodiments. It is intended that changes and modifications can be made to the described embodiments without departing from the true scope and spirit of the subject invention as defined by the appended claims.

### DETAILED DESCRIPTION OF THE INVENTION

This invention will be further understood in view of the following detailed description of exemplary embodiments. The system and methods described herein are useful for analyzing data obtained by the application of an ultrasound-generated harmonic load applied to a small tissue region.

An exemplary embodiment of a system is described herein, and includes signal or image acquisition equipment. For example, the apparatus as described above in Konofagou and Hynynen may be used to apply an oscillatory, internally applied radiation force to tissue by use of an ultrasound beam, thereby inducing local harmonic motion in such tissue. The equipment described herein detects the phase and amplitude of the applied oscillatory force and the phase and amplitude of the resulting motion of the tissue. The applied force and resulting displacements of the tissue may be written onto a tape, memory card, or

other medium by an appropriate recording device. Image processing equipment is used to process the data in accordance with the invention. Image processing may be performed by a personal computer, such as a Dell OptiPlex GX270 Small MiniTower, or other computer, having a central processing unit, an input device, such as tape drive, memory card slot, etc., for receiving the data and a keyboard for receiving user inputs, and an output device, such a monitor, a printer, or a recording device for writing the output onto a tape, memory card, or other medium. Image processing equipment may also located on several computers, which are operating in a single location or which are connected as a remote network.

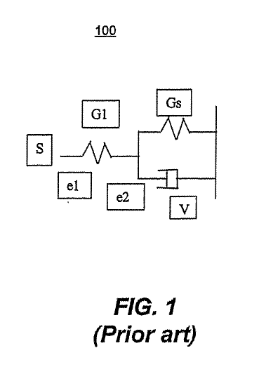

As illustrated in FIG. 1, the Maxwell model for viscoelasticity provides a short-time (or high-frequency) modulus, a long-time (or low frequency) modulus and a viscous component. The viscoelastic response can be characterized by the response to a sinusoidal strain input. For
S=S₀ sin(ωt),
the response of a viscoelastic material has an in-phase component (which tends to store energy) and an out-of phase component (which tends to dissipate energy):
ε=εᵢₙ sin(ωt)+εₒᵤₜ cos(ωt) (1)
This can also be expressed as
ε=ε₀ sin(ωt+φ) (2)
with φ the phase angle.

The Maxwell model can be approximated at higher frequencies by considering the viscous component as rigid, giving G=G₁. At lower frequencies, the effect of the viscous component can be ignored, giving G=G₁Gₛ/(G₁+Gₛ).

The effect of the viscous component is seen at intermediate frequencies. The Maxwell model gives an in-phase component which is:

$${ɛ}_{i n} = \frac{( {G}_{s}^{2} + {G}_{1} {G}_{s} + {ω}^{2} {V}^{2} ) S}{{G}_{1} ( {G}_{s}^{2} + {ω}^{2} {V}^{2} )} \qquad ( 3 )$$

and an out-of-phase component which is:

$${ɛ}_{out} = \frac{ω VS}{{G}_{s}^{2} + {ω}^{2} {V}^{2}} \qquad ( 4 )$$

The ratio of the two is usually taken as the phase angle:

$$tan ϕ = \frac{{ɛ}_{out}}{{ɛ}_{i n}} = \frac{{G}_{1} ω V}{{G}_{s}^{2} + {G}_{1} {G}_{s} + {ω}^{2} {V}^{2}} ; \qquad ( 5 )$$

which has a maximum at

$$ω = \frac{\sqrt{{G}_{s}^{2} + {G}_{1} {G}_{s}}}{V} . \qquad ( 6 )$$

Thus, given measurements for the high frequency modulus and the low frequency modulus, the frequency where the phase angle, or phase shift, is at a maximum can determine the viscosity parameter in the Maxwell model.

The finite element model used in accordance with an exemplary embodiment of the invention consists of a square region spanned by a 100-by-100 element mesh of linearly interpolated plane strain elements: The bottom edge was fixed, and the three other edges were stress-free. LS-Dyna was used for the calculations (LSTC, Livermore, Calif.), and a viscoelastic model was used in which the bulk modulus of the material was constant and the shear modulus, for a step input in stress, is given by:
G=Gₗ+(Gₛ−Gₗ)e^{-βt} (7)
with Gₗ the long-time modulus and Gₛ the short-time modulus. The coefficient β is the inverse of a relaxation time constant. According this exemplary embodiment, β was taken as 125. (In the case of the case of a purely elastic tissue, the coefficient β is 0.)

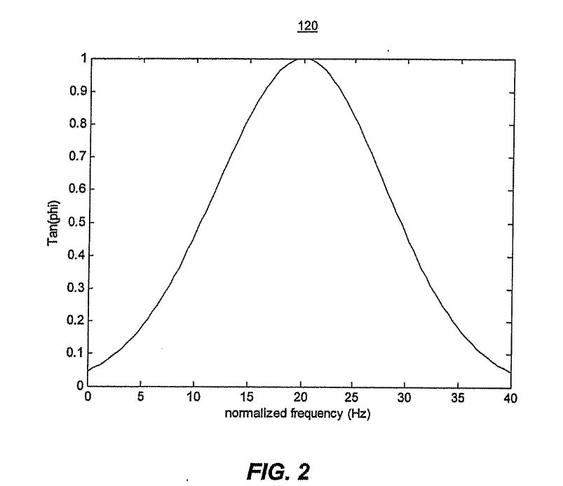

As illustrated in FIG. 2, frequencies of the input force ranging from 0.01 Hz to 40 Hz (at a step of 0.1 Hz) were used in order to determine the optimal range for the calculation of the viscosity V. A spectrum of phase angle was then obtained as a function of frequency, and it was determined in the exemplary embodiment, that an optimal range would be at a frequency about 20 Hz.

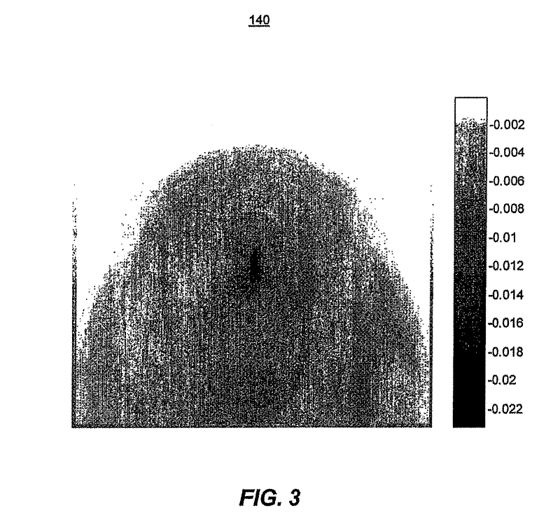

The central node in the region was loaded sinusoidally by a load that varies from zero to 0.001N in the downward direction at 20 Hz. A displacement image of the model is illustrated in FIG. 3.

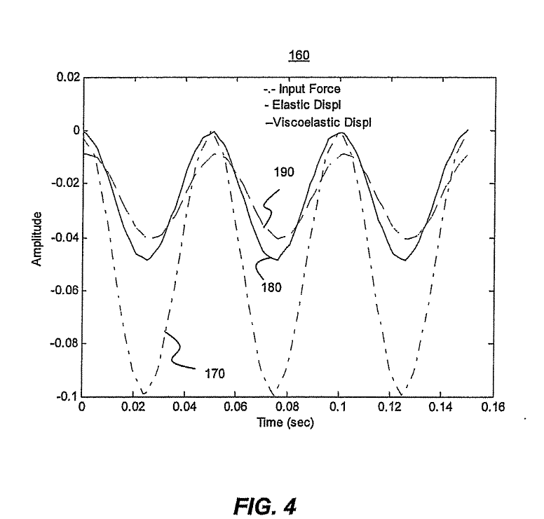

Dynamics were studied by time stepping, using the central difference method with a step size of 0.001. Since the load had a steady-state component, there was a transient consolidation. After 1 second of loading, the results for three loading cycles were collected at 0.005 second intervals. A time plot 160 is shown at FIG. 4, illustrating the input force 170, the elastic displacement 180, and the viscoelastic displacement 190.

As illustrated in FIG. 4, a phase shift between the applied force and the estimated displacement was obtained in the case of the viscoelastic model described herein. (Such phase shift is absent in the case of the elastic model, which did not take viscosity into account.) The phase shift was calculated by locating the maximum amplitude of the input force and the maximum amplitude of the output displacement and then taking their difference. An exemplary method is described in the Appendix herein.

According to equation 5, the two shear moduli, G₁ and Gₛ should be determined in order to determine the viscosity V from the phase shift. The moduli correspond to the cases at high frequency (f>20 Hz) and low frequency (f<20 Hz), where the viscoelastic tissue behaves elastically. More precisely, at high frequency only the first spring responds to the force (FIG. 1), and at low frequency the two springs act in series (FIG. 1). In order to determine those, the model was used at the frequencies of 40 Hz and 1 Hz, respectively.

By applying the Kelvin solution, i.e., the analytical solution for the application of a harmonically-applied point load force on an infinite medium, the two shear moduli, Gₗ and Gₛ, were determined through application of the solution and by using the input force and the output displacements at the corresponding frequencies. Finally, the phase shift was estimated using Eq. (5) and solving for the viscosity V.

Further exemplary embodiments of a system are discussed herein. According to these exemplary embodiments, the harmonic motion imaging (HMI) technique is used to estimate unidirectional tissue displacements remotely induced by the acoustic radiation force.

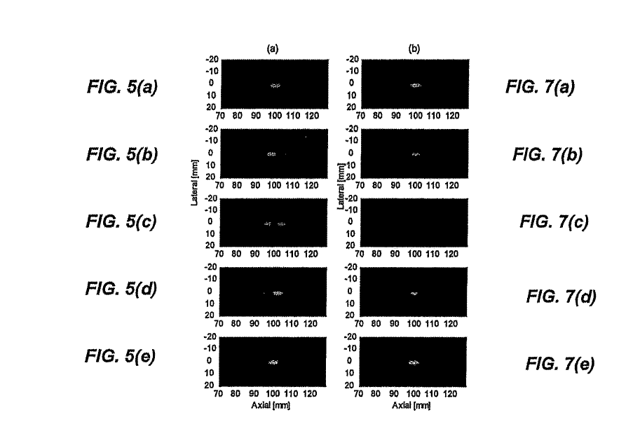

According one embodiment of the system, focused ultrasound therapy is provided using two separate focused ultrasound transducer elements working at different frequencies (f and f+Δf). The two overlapping focused beams produce an acoustic radiation force field moving at the difference frequency (Δf). The acoustic radiation force field produced by the two overlapping focused sound beams at two different frequencies Δf=50 Hz is illustrated in FIGS. 5(a)-5(e). Each of the suuccessive images in FIGS. 5(a)-5(b) was obtained 4 ms subsequent to the previous image.

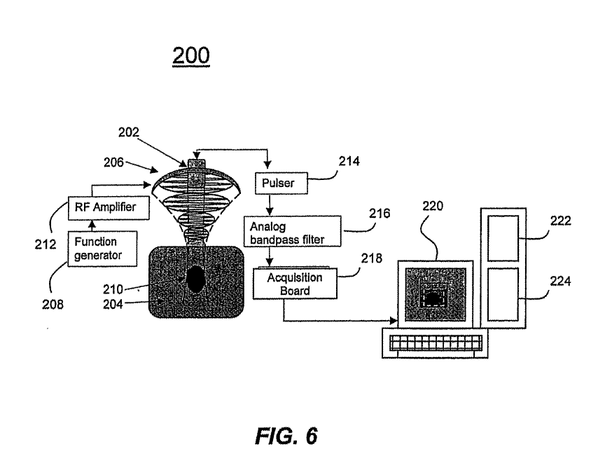

According to another exemplary embodiment of the system, designated system 200, and illustrated in FIG. 6, the harmonic radiation force is produced by a single focused ultrasound element or focused transducer 202 to determine viscoelastic properties of a medium 204, such as, e.g., body tissue, gel phantoms or bovine liver. The acoustic radiation force field of a single amplitude-modulated (“AM”) focused ultrasound beam is illustrated in FIGS. 7(a)-7(e), in which the images are taken at 4 ms intervals. When compared with the acoustic radiation force field produced by two overlapping focused ultrasound beams (FIGS. 5(a)-5(e), the spatial invariance of the radiation force field using the one-beam configurations (FIGS. 7(a)-7(e)) may be seen.

The displacements of the medium 204 or tissue may be measured at the same location of force application using a separate imaging transducer or diagnostic transducer 206. Using the methods and systems described herein, the displacements are measured during application of the acoustic radiation force, so that this method can be used for the monitoring of the mechanical properties of tissues during focused ultrasound (FUS) therapy.

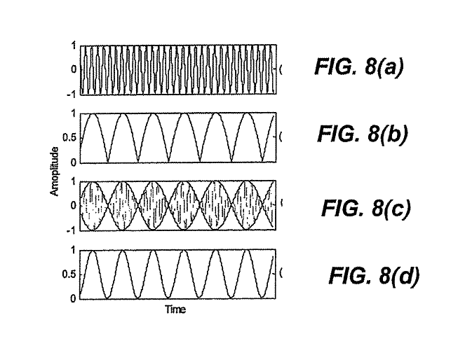

In the exemplary embodiment, an acoustic radiation force was generated by a 4.68 MHz focused transducer 202, using a low-frequency Amplitude-modulated (AM) radio frequency (RF) signal. A function generator 208 (for example, Agilent (HP) 33120A) may be used to produce the RF signal at 4.68 MHz, as illustrated in FIG. 8(a). The amplitude of the RF signal was then modulated in amplitude using a second function generator (not shown) that generates a low frequency modulation, as illustrated in FIG. 8(b). The focused transducer 202 may generate a pressure field shown in FIG. 8(c) and a modulated acoustic intensity at the focus 210, illustrated in FIG. 8(d). In the exemplary embodiment, amplitude-modulated (AM) frequencies were varied from about 10 Hz to about 100 Hz. The output of the function generator 212 could be adjusted from 100 mVpp to 600 mVpp and then amplified by an RF Amplifier 212, such as 50 dB RF-amplifier (EIN 3100L). The sonication time may be adjusted to induce oscillations (e.g., 100 oscillations) at the frequency of the modulation.

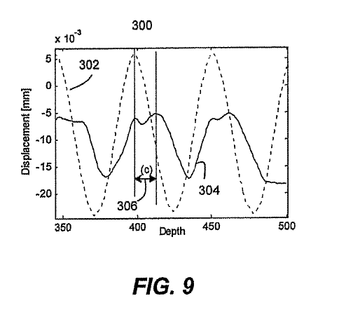

A 7.5 MHz single-element, diagnostic transducer 206 was placed through the center of the focused transducer 202 so that the diagnostic and focused beams may be properly aligned. A pulser 214 is provided. A bandpass analog filter 216 (e.g., Reactel, Inc.) may be used to remove the spectrum of the focused beam. Consecutive RF signals were acquired with a Pulse Repetition Frequency (PRF) of 5 kHz (Panametrics 5051PR). An acquisition board 218 (Gage Applied Technologies) was used to capture RF data with a sampling frequency 80 MHz. A 1D cross-correlation technique at a workstation 220 having a processor 222 and a memory 224 (e.g., Dell OptiPlex GX270 Small MiniTower) was used to calculate axial (along the ultrasound beam axis) displacements between two successive RF images, as is known in the art. (See, e.g., E. E. Konofagou, M. Ottensmeyer, S. Agabian, S. L. Dawson, K. Hynynen, “Estimating localized oscillatory tissue motion for assessment of the underlying mechanical modulus,” Ultrasonics, vol. 42, pp. 951-956, 2004, which is incorporated by reference in its entirety herein.) FIG. 9 illustrates a time plot 300 showing the normalized input radiation force intensity 302 and the output displacement 304. The amplitude of the displacement variation was measured, and a phase shift 306 was calculated between the amplitude-modulated signal 302 and the displacement 304.

### EXAMPLES

1. Finite Element Analysis

According to an exemplary embodiment, finite-element simulations (FEA) of a two-dimensional, plane strain three-layered model in lieu of actual test data were generated on Algor software (Algor, Inc, Pittsburgh, Pa.). The Young's modulus of the middle layer was allowed to change relative to the adjacent layers of fixed modulus equal to 10 kPa. In order to simulate the experimental application of HMI, a sinusoidal force of frequency equal to 200 Hz sequentially on each node of the FEA model. Simulated ultrasonic RF data were generated for each step of vibration and for each node using a convolutional model and the calculated displacements. Cross-correlation techniques using a 2 mm window and 80% overlap were applied on the RF data in order to image the incremental displacement in the direction of the applied force across the model. Eight different cases were studied; four of different moduli (5-40 kPa) and same relative viscous damping coefficient equal to 10 while the other four had different damping coefficients (0-10) and same modulus (40 kPa). The mean-squared amplitude and the phase shift of the estimated displacements relative to the applied force were studied in each FEA case in order to identify their distinct viscoelastic properties.

In M-mode HMI images, the amplitude of the displacements decreased exponentially with higher modulus and higher damping coefficient. However, only the increase of the viscous coefficient was shown to lead to a positive phase shift on the order of 50 microsec between the input radiation force and the resulting displacement. The estimated effect of the local viscous coefficient on the displacement amplitude was then removed so that the latter only indicated the effect of the change in modulus. Spatial HMI images were then generated indicating the regions of different elastic modulus and different viscous coefficients in all eight cases.

2. Tissue-Mimicking Phantom Experiments

A gelatin gel material was used for the tissue mimicking phantoms. Phantom preparation was completed according to T. J. Hall, M. Bilgen, M. F. Insana, T. Krouskop, “Phantom Materials for Elastography,” IEEE UFFC Trans. 44, 6, 1997, which is incorporated by reference in its entirety herein, Five homogeneous phantoms with different stiffness (20 kPa, 30 kPa, 40 kPa, 50 kPa, and 60 kPa) and a 20 kPa tissue mimicking phantom with a 40 kPa inclusion were made.

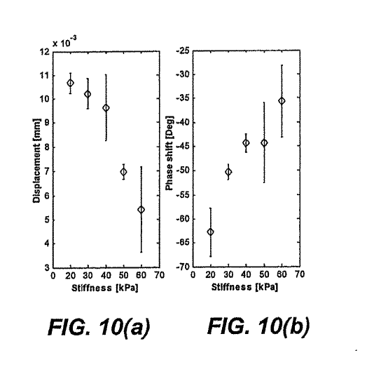

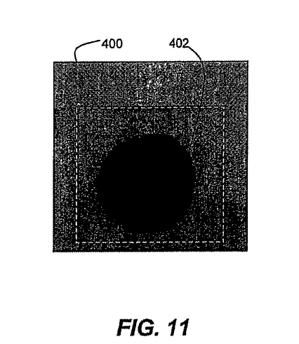

FIGS. 10(a) and 10(b) show results from the experiment. In order to create an effective acoustic radiation force, intensity of the focused beam used was 658.5 W/cm² at an AM frequency 50 Hz. The intensity of the focused beams is calculated according to:

$$I = \frac{{P}^{2}}{2 ρ c} \qquad ( 8 )$$

where, p=1.1 g/cm³, c=1.5·10⁵ cm/s and P=acoustic pressure at the focus for V-500 mVpp. The results indicate that displacement values decrease from 10.3 microns to 4.15 microns as gel stiffness increase from 20 kPa to 60 kPa. (FIG. 10(a).) The HMI displacements clearly indicate the stiffness variation. Using the system methods described herein, phase shifts were found to decrease from −66.4° to −30.4° consistent with increasing gels stiffness. (FIG. 10(b).)

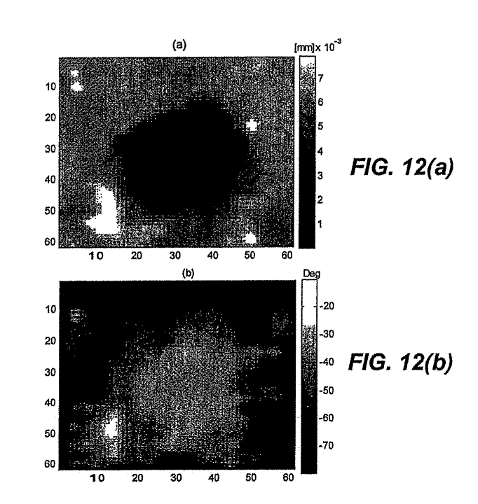

Secondly, the experiment was performed in a gelatin gel 400 having a stiffness of 20 kPa. The gel contained a cylindrical inclusion 402 having a stiffness of 40 kPa. (FIG. 1 1). The transducer was moved along a 2D grid using a computer-controlled positioner (e.g., Velmex, Inc.). A 20 mm×20 mm zone was raster-scanned with a step size of 1 mm. The 2D maps of displacement amplitude and phase shift are shown on the FIGS. 12(a) and 12(b), respectively. The average displacement in the inclusion 402 is 3.3 microns (FIG. 12(a)) and the phase shift is −34° (FIG. 12(b)). Using the system methods described herein, the average displacement in the background 400, i.e., the surrounding gelatin gel, was found to be 6.1 microns (FIG. 12(a)) and the phase shift to be −65.9° (FIG. 12(b)). These results are consistent with the inverse relationship between displacement and elastic modulus, as described. e.g., in E. E. Konofagou, J. Thierman, K. Hynynen, “A focused ultrasound method for simultaneous diagnostic and therapeutic applications-simulation study,” Phys. Med. Biol. vol. 46, pp. 2967-2984, 2001, which is incorporated by reference in its entirety herein. The phase shift relationship between amplitude-modulated input signal inducing the acoustic radiation force, and measured displacements can be used to estimate the viscoelastic properties in tissue-mimicking phantoms as well as in-vitro tissues.

3. Monitoring of FUS Ablation

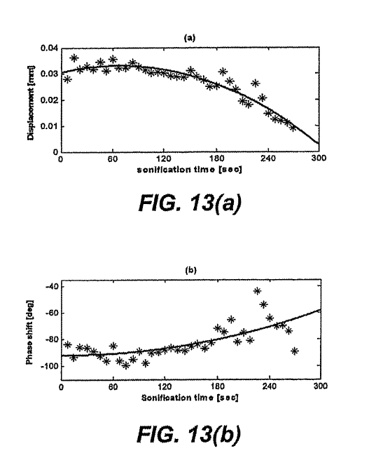

An ablation tissue experiment was performed on in-vitro tissue samples. A piece of 50 mm×50 mm bovine liver was submerged in degassed water. In this experiment, the intensity of the focused ultrasound beam was 948.23 W/cm² (calculated according to Equation [6] for V=600 m Vpp) at the focus 210, in order to simultaneously generate the harmonic radiation force and the tissue ablation. The frequency of the modulation was 50 Hz and the total sonication time was approximately 288 sec. The displacements were monitored in real-time. The variation of the amplitude and phase shift are shown on FIGS. 13(a) and 13(b), respectively. The oscillatory displacement amplitude and displacement-force phase shift start to rapidly decrease beyond 120 seconds of continuous sonication, possibly indicating tissue coagulation or lesion formation beyond the sonication period. The effect on the echoes induced by the change of the speed of sound with temperature induces a low frequency shift of the speckle, and was successfully separated from the higher frequency displacements induced by the harmonic radiation force. This technique is able to follow the heating process and detect the time of coagulation.

While there have been described what are believed to be the preferred embodiments of the present invention, those skilled in the art will recognize that other and further changes and modifications may be made thereto without departing from the spirit of the invention, and it is intended to claim all such changes and modifications as fall within the true scope of the invention.

APPENDIX
A portion of the disclosure of this patent document contains material
which is subject to copyright protection. The copyright owner has no
objection to the facsimile reproduction by anyone of any portion of the
patent document, as it appears in any patent granted from the present
application or in the Patent and Trademark Office file or records available
to the public, but otherwise reserves all copyright rights whatsoever.
phaseshift.m: a Matlab function that calculates the phase shift
between the input force and the resulting displacement
function shift=phaseshift(force, disp);
[maxf,locf] = max(force);
[maxd,locd] = max(disp);
shift=locf−locd;

What is claimed is:

1. A method for imaging the localized viscoelastic properties of tissue comprising:
inducing localized oscillatory motion of tissue through the application of an oscillatory radiation force having a phase and amplitude;
detecting the phase and amplitude of the induced localized oscillatory motion of the tissue induced by the application of the oscillatory radiation force; and
determining the viscous properties of the tissue by calculation of a phase shift between the applied oscillatory radiation force and the induced localized oscillatory motion of the tissue.

2. The method as recited in claim 1, wherein inducing localized oscillatory motion of tissue comprises applying an oscillatory ultrasound radiation force.

3. The method as recited in claim 2, wherein inducing localized oscillatory motion of tissue comprises applying two overlapping focused ultrasound beams.

4. The method as recited in claim 2, wherein inducing localized oscillatory motion of tissue comprises applying one focused ultrasound beam.

5. The method as recited in claim 4, wherein inducing localized oscillatory motion of tissue comprises applying one amplitude-modulated ultrasound beam.

6. A method for imaging the localized viscoelastic properties of tissue comprising:
receiving a first signal representative of an applied oscillatory radiation force having a phase and amplitude;
receiving a second signal representative of an induced localized oscillatory motion of the tissue induced by the application of the oscillatory radiation force, the second signal having a phase and amplitude; and
determining the viscous properties of the tissue by calculation of a phase shift between the applied oscillatory radiation force and the induced localized oscillatory motion of the tissue.

7. The method as recited in claim 6, wherein receiving the second signal representative of an induced localized oscillatory motion of the tissue comprises receiving successive images of the tissue and determining axial displacements of tissue from the successive images of the tissue.

8. The method as recited in claim 6, further comprising, prior to receiving the first signal, inducing localized oscillatory motion of tissue.

9. The method as recited in claim 8, wherein inducing localized oscillatory motion of tissue comprises applying an oscillatory ultrasound radiation force.

10. The method as recited in claim 9, wherein inducing localized oscillatory motion of tissue comprises applying two overlapping focused ultrasound beams.

11. The method as recited in claim 9, wherein inducing localized oscillatory motion of tissue comprises applying one focused ultrasound beam.

12. The method as recited in claim 1 1, wherein inducing localized oscillatory motion of tissue comprises applying one amplitude-modulated ultrasound beam.

13. A system for imaging the localized viscoelastic properties of tissue comprising:
a first transducer inducing localized oscillatory motion of tissue through the application of an oscillatory radiation force having a phase and amplitude;
a second transducer detecting a phase and amplitude of the induced localized oscillatory motion of the tissue induced by the application of the oscillatory radiation force; and
a processor and a memory operatively coupled to the processor, the memory storing program instructions for execution by the processor to determine the viscous properties of the tissue by calculation of a phase shift between the applied oscillatory radiation force and the induced localized oscillatory motion of the tissue.

14. The system as recited in claim 13, wherein the first transducer applies an oscillatory ultrasound radiation force.

15. The system as recited in claim 14, wherein the first transducer applies one amplitude-modulated ultrasound beam.

16. A system for imaging the localized viscoelastic properties of tissue comprising:
a processor and a memory operatively coupled to the processor, the memory storing program instructions for execution by the processor to receive a first signal representative of an applied oscillatory radiation force having a phase and amplitude, to receive a second signal representative of an induced localized oscillatory motion of the tissue simultaneous with the application of the oscillatory radiation force, the second signal having a phase and amplitude, and to determine the viscous properties of the tissue by calculation of a phase shift between the applied oscillatory radiation force and the induced localized oscillatory motion of the tissue.

17. The system as recited in claim 16, wherein the processor is further adapted to determine axial displacements of tissue from successive images of the tissue.

18. The system as recited in claim 16, further comprising a first transducer inducing localized oscillatory motion of tissue through the application of the oscillatory radiation force.

19. The system as recited in claim 18, wherein the first transducer applies one amplitude modulated ultrasound beam.

20. The system as recited in claim 16, further comprising a second transducer detecting a phase and amplitude of the induced localized oscillatory motion of the tissue simultaneous with the application of the oscillatory radiation force.
